(19) United States
(12) Patent Application Publication
(10) Pub. No.: US 2021/0231651 A1
(43) Pub. Date: Jul. 29, 2021

(54) METHODS AND COMPOSITIONS FOR POINT OF CARE MEASUREMENT OF THE BIOAVAILABILITY OF THERAPEUTIC BIOLOGICS

(71) Applicant: GENALYTE, INC., San Diego, CA (US)

(72) Inventors: David BROWN, Oceanside, CA (US); Martin Anthony GLEESON, San Diego, CA (US)

(73) Assignee: Genalyte, Inc., San Diego, CA (US)

(21) Appl. No.: 17/051,278

(22) PCT Filed: Apr. 29, 2019
(86) PCT No.: PCT/US2019/029740

§ 371 Date: Oct. 28, 2020

Related U.S. Application Data
(60) Provisional application No. 62/664,633, filed on Apr. 30, 2018.

(51) Int. Cl. G01N 33/543

(52) U.S. Cl. CPC G01N 33/54306; G01N 33/54386

(57) ABSTRACT

Embodiments provided herein relate to methods, compositions and systems for rapid determination of the bioavailability of a therapeutic biologic in a sample from a subject. In some such embodiments, the presence or absence of a therapeutic biologic in a sample can be determined using an optical sensor

### RELATED APPLICATIONS

This application claims priority to U.S. Prov. App. No. 62/664,633 filed Apr. 30, 2019 entitled “METHODS AND COMPOSITIONS FOR POINT OF CARE MEASUREMENT FOR BIOAVAILABILITY OF BIOLOGICS” which is incorporated by reference in its entirety.

### FIELD OF THE INVENTION

Some embodiments provided herein relate to methods, compositions and systems for rapid determination of the bioavailability of a therapeutic biologic in a sample from a subject. In some such embodiments, the presence or absence of a therapeutic biologic in a sample can be determined using an optical sensor.

### BACKGROUND OF THE INVENTION

Autoimmune disorders are a significant and widespread medical problem. For example, rheumatoid arthritis is an autoimmune disease affecting more than two million people in the United States. Rheumatoid arthritis causes chronic inflammation of the joints and typically is a progressive illness that has the potential to cause joint destruction and functional disability. Traditional treatments for the management of rheumatoid arthritis and other autoimmune disorders can include administration of drugs which can reduce pain and inflammation, and drugs which promote disease remission and prevent progressive joint destruction may also be administered to patients, such drugs include hydrochloroquine, azulfidine and immunosuppressive agents, such as methotrexate, azathioprine, cyclophosphamide, chlorambucil and cyclosporine. However, many of these drugs can have detrimental side-effects. Thus, additional therapies for rheumatoid arthritis and other autoimmune disorders have been sought.

Tumor necrosis factor alpha (TNFα) is a cytokine produced by numerous cell types and has been implicated in the pathophysiology of a variety of other human diseases and disorders, including shock, sepsis, infections, autoimmune diseases, rheumatoid arthritis, Crohn's disease, transplant rejection and graft-versus-host disease. Therapeutic strategies have been designed to inhibit or counteract TNFα activity. For example, therapeutic biologics that include antibodies that bind and neutralize TNFα have been sought as a means to inhibit TNFα activity. Four TNFα inhibitors including infliximab which is a chimeric anti-TNFα monoclonal antibody, etanercept which is a TNFR-Ig Fc fusion protein, adalimumab which is a human anti-TNFα monoclonal antibody, and certolizumab pegol which is a PEGylated Fab fragment, have been approved by the FDA for treatment of rheumatoid arthritis. While such therapeutic biologics have demonstrated success in the treatment of rheumatoid arthritis and other autoimmune disorders, not all subjects treated respond, or respond well, to such therapy. In some cases, administration of a TNFα inhibitor can induce an immune response to a therapeutic biologic and lead to the production of autoantibodies in a patient. Such autoantibodies can be associated with hypersensitive reactions and dramatic changes in pharmacokinetics and bioavailability that precludes further treatment with the therapeutic biologic. Assays to determine the presence of autoantibodies against a therapeutic biologic in a patient can be costly and time consuming. Moreover, other clearance mechanisms in a patient can reduce the bioavailability of a therapeutic biologic in a subject. Thus, there is a need to rapidly and effectively determine the bioavailability of a therapeutic biologic in a subject.

### SUMMARY OF THE INVENTION

Some embodiments include a method of determining the bioavailability of a biologic in a subject comprising: (a) providing a sample obtained from a subject treated with a biologic; (b) contacting a first capture probe attached to an optical sensor with the sample, wherein the biologic selectively binds to the first capture probe; (c) contacting the biologic bound to the first capture probe with a second capture probe, wherein the second capture probe selectively binds to the biologic; and (d) measuring a change, no substantial change, or no change in one or more resonance wavelengths at the optical sensor, thereby detecting the presence or absence of the biologic in the sample.

In some embodiments, the subject has been administered the biologic more than seven days before the sample was provided. In some embodiments, the subject was administered the biologic more than twelve days before the sample was provided. In some embodiments, the subject was administered the biologic more than 2 weeks before the sample was provided.

In some embodiments, no change, or no substantial change in one or more resonance wavelengths at the optical sensor is detected. In some embodiments, no change, or no substantial change in one or more resonance wavelengths at the optical sensor is indicative of the biologic lacking bioavailability for the subject.

In some embodiments, a change in one or more resonance wavelengths at the optical sensor is detected. In some embodiments, a change in one or more resonance wavelengths at the optical sensor is indicative of the biologic having bioavailability for the subject. In some embodiments, the detected change in one or more resonance wavelengths at the optical sensor is indicative of the level of the biologic in the sample.

Some embodiments also include determining the presence or absence of an antibody against the biologic in the sample.

In some embodiments, the biologic comprises an antigen-binding protein. In some embodiments, the biologic is capable of binding the first capture probe in vivo. In some embodiments, the biologic comprises a human IgG polypeptide constant region selected from a light chain, and a heavy chain. In some embodiments, the biologic comprises a human IgG1 constant region selected from a light chain, and a heavy chain. In some embodiments, the biologic is selected from the group consisting of infliximab, abatacept, adalimumab, alefacept, etanercept, trastuzumab, ustekinumab, golimumab, and certolizumab pegol.

In some embodiments, the first capture probe comprises a polypeptide selected from the group consisting of TNFα, CD80, CD86, CD2, HER2, IL-12, IL-23, and a fragment of any one of the foregoing polypeptides which selectively binds to the biologic. In some embodiments, the first capture probe comprises TNFα.

In some embodiments, the second capture probe comprises an anti-human IgG antibody or antigen-binding fragment thereof.

In some embodiments, the sample comprises serum.

In some embodiments, the subject is mammalian. In some embodiments, the subject is human.

In some embodiments, the first capture probe comprises TNFα, the biologic comprises infliximab or adalimumab, and the second capture probe comprises an anti-human IgG antibody or antigen-binding fragment thereof.

In some embodiments, the amount of the first capture probe attached to the optical sensor is from about 10 μg to about 300 μg.

In some embodiments, contacting a first capture probe attached to an optical sensor with the sample comprises flowing the sample over the optical sensor for about 3 minutes at a rate of about 40 μl/min.

In some embodiments, contacting the biologic bound to the first capture probe with a second capture probe comprises flowing a buffer comprising the second capture probe over the attached biologic for about 3 minutes at a rate of about 30 μl/min.

Some embodiments also include flowing a buffer over the optical sensor for about 1.5 minutes at a rate of about 40 μl/min prior to contacting a first capture probe attached to an optical sensor with the sample.

Some embodiments also include flowing a buffer over the optical sensor for about 2 minutes at a rate of about 40 μl/min after contacting a first capture probe attached to an optical sensor with the sample.

In some embodiments, a baseline determination of one or more resonance wavelengths at the optical sensor is made. In some embodiments, the baseline determination of resonance wavelengths is made during the final 30 seconds of a step selected from: the flowing a buffer over the optical sensor for about 1.5 minutes at a rate of about 40 μl/min prior to contacting a first capture probe attached to an optical sensor with the sample, the flowing a buffer over the optical sensor for about 2 minutes at a rate of about 40 μl/min after contacting a first capture probe attached to an optical sensor with the sample, and the flowing a buffer comprising the second capture probe over the attached biologic.

In some embodiments, a flow cell comprises the optical sensor.

Some embodiments also include a system for detecting a biologic comprising: an optical sensor comprising: a ring resonator, a first capture probe attached to the ring resonator, wherein the first capture probe selectively binds to a therapeutic biologic, and a second capture probe which selectively binds to the therapeutic biologic. Some embodiments also include a detector adapted to measure a change in one or more resonance wavelengths at the optical sensor.

Some embodiments also include an assay for determining the presence of an antibody against the therapeutic biologic in a sample, the assay comprising: a third capture probe which selectively binds to the therapeutic biologic or to the second capture probe, wherein the third capture probe comprises a first binding partner; and a fourth capture probe which selectively binds to the third capture probe, wherein the fourth capture probe comprises a second binding partner.

In some embodiments, the first or second binding partner is selected from biotin and streptavidin.

In some embodiments, the first capture probe comprises TNFα, the therapeutic biologic comprises infliximab or adalimumab, and the second capture probe comprises an anti-human IgG antibody or antigen-binding fragment thereof.

In some embodiments, the first capture probe is attached to the optical sensor at a concentration from about 10 μg to about 300 μg.

In some embodiments, a flow cell comprises the optical sensor.

### BRIEF DESCRIPTION OF THE DRAWINGS

FIG. 1 shows a schematic diagram of an optical sensor comprising a waveguide and a ring resonator. FIG. 1 schematically illustrates the range of wavelengths that may be input into the optical sensor and the resultant spectral output of the optical sensor. A decrease in the optical output at the resonance frequency of the ring resonator is visible in the output spectrum shown

FIG. 2 is a cut-away view of a waveguide schematically showing an intensity distribution having an evanescent tail extending outside the waveguide where an element such as a molecule or particle may be located so as to affect the index of refraction of the waveguide.

FIG. 3 depicts a graph of signal from waveguides functionalized with TNFα in GENLYTE relative units (GRU) for various serum samples from patients that had been treated with Adalimumab (HUMIRA).

### DETAILED DESCRIPTION

Some embodiments provided herein relate to methods, compositions and systems for rapid determination of the bioavailability of a therapeutic biologic in a sample from a subject. In some such embodiments, the presence or absence of a therapeutic biologic in a sample can be determined using an optical sensor. Embodiments provided herein relate to determining the bioavailability of therapeutic biologics that have been administered to a subject. Biologics include large complex molecules, such as proteins, used to improve health through manipulation of natural and disease biological processes. During treatment with therapeutic biologics, an immune response against the therapeutic biologic can be raised in a subject, and the subject can often develop antibodies against the biologic. As a result, the efficacy of the biologic can be limited by a variety of mechanisms including steric blocking which limits the ability of the biologic to bind to its target in a subject, and reduction in the amount of biologic present in a subject due to more rapid clearance. Some embodiments provided herein relate to the rapid determination of the bioavailability of a biologic for a subject. In some embodiments a device for such determinations can be handheld. Embodiments provided herein can allow a clinician to test a patient while in the office at time of dosing to determine whether the biologic in the subject's blood is available to interact with its target. Advantageously, a clinician can identify non-responders, avoid administering ineffective drug, and instead provide an alternative therapy. In some embodiments, a test is provided that screens for binding of drug to target which can provide an actionable result at time of testing and so aid therapeutic decision making.

Optical ring resonance is a property of light that yields the removal of specific wavelengths when light enters a circular waveguide, called a ring resonator. Specifically, wavelengths of light that are exactly equal to the circumference of the ring divided by an integer, times the refractive index of the surrounding media, will become trapped and resonate within the ring, while all other wavelengths of light can leave the resonator (Luchansky M. S., et al. 2010 Biosens. Bioelectron. 26:1283-1291 which is incorporated by reference in its entirety). The resonant wavelengths that are trapped in the ring leave a negative peak in the spectrum of light leaving the ring. The waveguide can be made in such a way that a portion of the light energy extends beyond the surface of the waveguide in the form of an evanescent tail that interacts with the material in the immediate proximity of the waveguide. Any matter that changes the index of refraction will change the resonant wavelengths in the ring resonator. It follows that when the refractive index of the surrounding media changes, the wavelengths of light that remain trapped in the ring resonator will change accordingly. The resonant wavelengths will shift proportionately higher as more matter is deposited above the ring. Thus, binding of material including protein and DNA can be detected directly since they have higher refractive indices than water. To enhance and amplify the signal, polystyrene beads or enzymatic deposition of an insoluble precipitate above the rings can be used.

Optical ring resonance is a phenomenon that occurs when an optical waveguide laid in the form of a ring is excited by a feed waveguide in close proximity and where the interaction region between the two waveguides is precisely controlled. In such a setting, evanescent coupling between the feed and ring waveguides allows a near complete energy transfer to the ring when wavelength of passing light is tuned to a resonant wavelength of the system. The device resonates under the condition m=2πrn_{eff}/λₘ where m is an integer, r is the radius of the ring, n_{eff} is the effective index of refraction of the waveguide, and λₘ is the resonant wavelength (see, e.g., Iqbal, M., et al., (2010) Label-free biosensor arrays based on silicon ring resonators and high-speed optical scanning instrumentation. IEEE J. Sel. Quantum Elec. 16 (3) which is incorporated by reference in its entirety). The phenomenon is typically observed by recording the spectrum (intensity versus wavelength) at the output of feed waveguide. For a system where excitation wavelengths are continuously tuned, the resulting spectrum will depict inverted peaks (troughs) in pass-through intensity at intervals governed by the resonant condition described above. In other words, all light is trapped in the ring waveguide for the duration that the light source dwells at a resonant wavelength. Cavity quality factor or Q enhances the sensitivity of the ring to changes in its immediate surroundings. Any disturbances such as bulk fluid exchange or molecular binding activity on its surface has the effect of changing n_{eff}, which in turn displaces the resonance wavelength. This property of the ring resonator lends itself for application in bio-sensing. Controlled light-matter interaction is achieved by pre-depositing a capture probe molecule on the ring's surface. During the assay, specific binding between this probe and a target analyte has the effect of changing n_{eff} by displacing lower refractive index water with a denser and more massive molecular complex. As mentioned, this induces a shift in one or more resonant wavelengths of the ring, which is recorded as a direct measure of binding activity. The interaction between light trapped inside the ring waveguide and macromolecules binding to the surface above the ring is achieved through the portion of the evanescent tail of the optical mode that is propagating outside the waveguide. As such, the shift in one or more resonant wavelengths is directly proportional to the amount of mass captured by probing molecules. In other words, the one or more resonant wavelengths will shift proportionately higher as more matter is deposited above the ring.

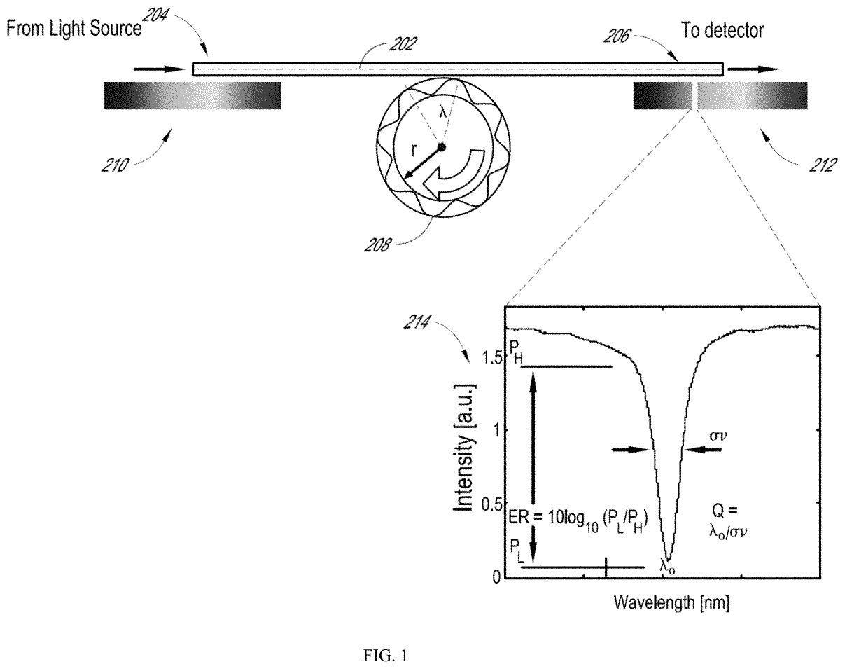

The operation of a ring resonator is shown in connection with FIG. 1. In this configuration, the optical sensor comprises an input/output waveguide 202 having an input 204 and an output 206 and a ring resonator 208 disposed in proximity to a portion of the input/output waveguide 202 that is arranged between the input 204 and the output 206. The close proximity facilitates optical coupling between the input/output waveguide 202 and the ring resonator 208, which is also a waveguide. In this example, the input/output waveguide 202 is linear and the ring resonator 208 is circular such that light propagating in the input/output waveguide 202 from the input 204 to the output 206 is coupled into the ring resonator 208 and circulates therein. Other shapes for the input/output waveguide 202 (for example, curved) and ring resonator 208 (e.g., oval, elliptical, triangular, etc.) are also possible.

FIG. 1 shows an input spectrum 210 to represent that the light injected into the waveguide input 204 includes a range of wavelengths, for example, from a narrow band light source having a narrow band peak that is swept over time (or from a broadband light source such as a super-luminescent diode). Similarly, an output spectrum 212 is shown at the waveguide output 206. A portion of this output spectrum 212 is expanded into a plot of intensity versus wavelength 214 and shows a dip or notch in the spectral distribution at the resonance wavelength, λ₀, of the ring resonator 208.

Without subscribing to any particular scientific theory, light “resonates” in the ring resonator when the number of wavelengths around the ring (e.g. circumference) is exactly an integer. In this example, for instance, at particular wavelengths, light circulating in the ring resonator 208 is at an optical resonance when:

mλ=2πrn
where m is an integer, λ is the wavelength of light, r is the ring radius, and n is the refractive index.

In this resonance condition, light circulating in the ring interferes with light propagating within the linear waveguide 202 such that optical intensity at the waveguide output 206 is reduced. Accordingly, this resonance will be measured as an attenuation in the light intensity transmitted down the linear waveguide 202 past the ring resonator 208 as the wavelength is swept by the light source in a manner such as shown in the plot 214 of FIG. 1.

Notably, the plot 214 in FIG. 1 shows the dip or notch having a width, δυ as measured at full width half maximum (FWHM) and an associated cavity Q or quality factor, Q=λ₀/δυ. The ring resonator 208 produces a relatively high cavity Q and associated extinction ratio (ER) that causes the optical sensor 104 to have a heightened sensitivity.

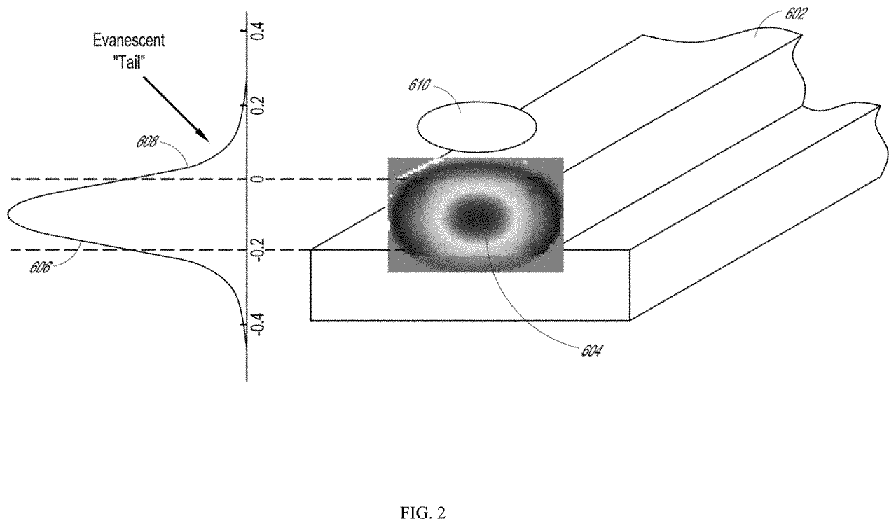

As is well known, light propagates within waveguides via total internal reflection. The waveguide supports modes that yield a spatially varying intensity pattern across the waveguide. A cross-section of a waveguide 602 shown in FIG. 2 illustrates an example intensity distribution 604. A plot 606 of the intensity distribution at different heights is provided adjacent the waveguide structure 602. As illustrated, a portion 608 of the electric field and optical energy referred to as the evanescent “tail” lies outside the bounds of the waveguide 602. The length of this field 608, as measured from the 1/e point, is between 50 and 150 nm, e.g. about 100 nm in some cases. An object 610 located close to the waveguide 602, for example, within this evanescent field length affects the waveguide. In particular, objects 610 within this close proximity to the waveguide 602 affect the index of refraction of the waveguide. The index of refraction, n, can thus be different when such an object 610 is closely adhered to the waveguide 602 or not. In various embodiments, for example, the presence of an object 610 increases the refractive index of the waveguide 602. In this manner, the optical sensor may be perturbed by the presence of an object 610 in the vicinity of the waveguide structure 602 thereby enabling detection. In various embodiments, the size of the object is about the length (e.g. 1/e distance) of the evanescent field to enhance interaction therebetween.

In the case of the ring resonator, an increase in the refractive index, n, increases the optical path length traveled by light circulating about the ring. Longer wavelengths can resonate in the resonator and, hence, the resonance frequency is shifted to a lower frequency. The shift in the one or more resonant wavelengths of the resonator can therefore be monitored to determine if an object 610 has located itself within close proximity to the optical sensor (e.g., the ring resonator and/or a region of the linear waveguide closest to the ring resonator). A binding event, wherein an object 610 binds to the surface of the optical sensor can thus be detected by obtaining the spectral output from the waveguide output and identifying one or more dips in intensity (or peaks in attenuation) therein and the shift of these one or more dips in intensity.

In various embodiments, the waveguide 602, e.g., the linear waveguide and/or the ring resonator comprise silicon. In some embodiments, the surface of the waveguide 602 may be natively passivated with silicon dioxide. As a result, standard siloxane chemistry may be an effective method for introducing various reactive moieties to the waveguide 602, which are then subsequently used to covalently immobilize biomolecules via a range of standard bioconjugate reactions.

Moreover, the linear waveguide, ring resonator, and/or additional on-chip optics may be easily fabricated on relatively cheap silicon-on-insulator (SOI) wafers using well established semiconductor fabrication methods, which are extremely scalable, cost effective, and highly reproducible. Additionally, these devices may be easily fabricated and complications due to vibration are reduced when compared to “freestanding” cavities. In one example embodiment, 8″ SOI wafers may each contain about 40,000 individually addressable ring resonators. One advantage of using silicon-based technology is that various embodiments may operate in the Si transparency window of around 1.55 μm, a common optical telecommunications wavelength, meaning that lasers and detectors are readily available in the commercial marketplace as plug-and-play components.

Some embodiments of the waveguides useful with the methods, systems and compositions provided herein include strip and rib waveguides. Other types of waveguides, such as for example, strip-loaded waveguides can also be used. Lower cladding lies beneath the waveguides. In some embodiments, the waveguides are formed from a silicon-on-insulator chip, wherein the silicon is patterned to form the waveguides and the insulator beneath provides the lower cladding. In many of these embodiments, the silicon-on-insulator chip further includes a silicon substrate. Details on the fabrication of silicon biosensor chips can be found in Washburn, A. L., L. C. Gunn, and R. C. Bailey, Analytical Chemistry, 2009, 81(22): p. 9499-9506, and in Bailey, R. C., Washburn, A. L., Qavi, A. J., Iqbal, M., Gleeson, M., Tybor, F., Gunn, L. C. Proceedings of SPIE—The International Society for Optical Engineering, 2009, the disclosures of which are hereby incorporated by reference in their entirety.

Still other designs than those specifically shown in the drawings herein may be employed. More ring resonators may be added. The resonators may also have different sizes and/or shapes. Additionally, the ring resonator(s) may be positioned differently with respect to each other as well as with respect to the input/output waveguide. Likewise, more non-ring resonator waveguides may be added.

In various embodiments, for example, a drop configuration is used. For example, in some such embodiments, a ring resonator is disposed between first and second waveguides. Light (such as a wavelength component) may be directed into an input of the first waveguide and depending on the state of the ring resonator, may be directed to either an output of the first waveguide or an output of the second waveguide. For example, for one or more resonant wavelengths, the light may be output from the second waveguide instead of the first waveguide. An optical detector may thus monitor shifts in intensity peaks to determine the presence of an analyte of interest detected by the optical sensor in some such embodiments.

Combinations of these different features are also possible. Moreover, multiple resonators and/or waveguides may be placed in any desired geometric arrangement. Additionally, spacing between resonators and/or waveguides may be varied as desired. Different features can be combined in different ways.

Also, although linear waveguides are shown in FIGS. 1 and 2 as providing access to the ring resonators, these waveguides need not be restricted to plain linear geometry. In some examples, for instance, these waveguides may be curved or otherwise shaped differently. Likewise the ring resonators need not be circularly shaped but can have other shapes. The ring resonators may be oval or elliptically-shaped, triangularly-shaped or irregularly shaped.

Other geometries may possibly be used for the resonator, such as, for example, microsphere, microdisk, and microtoroid structures. See, e.g., Vahala, Nature 2003, 424, 839-846; and in Vollmer & Arnold, Nature Methods 2008, 5, 591-596, the disclosures of which are hereby incorporated by reference in their entirety. Again, combinations of these different features are also possible and different features can be combined in different ways.

Additional details regarding sensors and apparatus for interrogating such sensors are included in U.S. Patent Publication 2011/0045472 titled “Monitoring Enzymatic Process” as well as PCT Publication WO 2010/062627 titled “Biosensors Based on Optical Probing and Sensing”, which are each incorporated herein by reference in its entirety.

An example system useful with the methods, systems and compositions provided herein includes a MAVERICK detection system (GENLYTE, INC. San Diego Calif.). See e.g., Mudumba S. et al., 2017 J. Immun. Methods 448:34-43 which is incorporated by reference herein in its entirety. The MAVERICK detection system automatically runs up to 12 chips one after the other, each chip including several waveguides.

### Silicon Chips

Some embodiments of the methods, systems and compositions provided herein include silicon chips. In some such embodiments, a 4×6 mm silicon chip can be manufactured in the same fabrication plant that manufactures computer chips, and designed to manipulate light rather than electricity, that is photons can be directed through the chip instead of electrons. In some embodiments, 136 rings are etched onto the chip. In some implementations, each ring can be 30 μm in diameter. In some cases, eight can be used as controls and remain covered by a Teflon-like per-fluoropolymer that can coat most of the chip. The other 128 rings can be organized into 32 clusters of 4 rings each, which may not be covered and so can be exposed to the material that flows above them. These rings can be functionalized by spotting test specific reagents on to them, which act as analyte specific capture probes. Thus, each test can be run in quadruplicate. The clusters can be organized in 2 rows with 16 clusters in each row. The gasket covering each chip can have a flow channel for each row of clusters. Thus, 2 samples can be tested for 16 assays each, or 1 sample can be tested on 32 assays in the current version of the chip. When the assay is being performed, light can enter the chip, for example, through grating couplers, travel through waveguides to the ring resonators, and then can return to the grating couplers where the intensity at each wavelength of the returning light can be measured. Negative peaks in the intensity of light can indicate the resonant wavelengths, and the shift in the wavelengths of the negative peaks can indicate a change in the refractive index above the ring cluster, which in turn can be proportional to the mass that has bound to the reagent above the cluster. The measured signal is the number of picometers (pm) the resonance peak shifts during the course of the assay. The shift in each ring can be measured separately, and the average of the shifts from the 4 rings in the cluster can be output as Genalyte Response Units (GRU). In some implementations, an outlier within a cluster can be removed automatically by software by applying Chauvenet's criterion if it is N3 standard deviations from the mean. The systems and approaches described above are just some examples. Other approaches, configurations, and arrangements that are different are possible.

Spotting the Chips with Reagents

Some embodiments of the methods, systems and compositions provided herein include spotting the chips with reagents, such as a first capture probe which selectively binds to a biologic. In some embodiments, spotting can include depositing a volume of a reagent on a substrate. In some embodiments, placing analytes onto a silicon chip can be done by using a spotter, such as a sciFLEXARRAYER S5 (Scienion, AG, Berlin). The Scienion spotter uses a glass piezo-electric nozzle (PDC-70), which dispenses 280-360 μl per drop. The on-board camera can ensure that each drop is precisely placed on a cluster by using real time image recognition of fiducial marks on each chip and drop trajectory after each dispense. The spotter can be enclosed in a glass chamber with humidity control to minimize static charge and to keep the spotted analytes from drying too quickly. Other techniques for activating the silicon surface of chips to allow binding of biological macromolecules have been described. See e.g., Kirk et al., (2013) Zwitterionic polymer-modified silicon microring resonators for label-free biosensing in undiluted human plasma. Biosens. Bioelectron. 42, which is incorporated by reference in its entirety.

### Gaskets and Reagents for Arrays

Some embodiments of the methods, systems and compositions provided herein include the use of certain gaskets and reagents. In some embodiments, up to 12 functionalized chips can be placed into a gasket that holds the chips in place so that they can be exposed to the scanning instrumentation of a system, such as a MAVERICK system described herein. Aspiration tubes in the gasket can allow liquid to be drawn up from the sample wells, and microfluidic channels can allow the reagents to flow over the chips and eventually go into a waste container. The gaskets can be paired with a 96 well reagent plate that contains diluted samples, wash buffers and amplification reagents. Since there are 12 chips per gasket, 2 channels per chip and up to 16 tests per channel, 384 tests can be performed on the chips in one array. In some embodiments, such tests can be organized so that different samples can be run on each channel, allowing 24 samples to be tested on 16 assays each. In some embodiments, different reagents could be spotted on every cluster on every chip, allowing 1 sample to be tested on 384 different assays.

### MAVERICK Systems

Some embodiments of the methods, systems and compositions provided herein include the use of a MAVERICK system. The MAVERICK system is designed to automate the procedure of running assays once the array with the functionalized chips and the reagent plate with the diluted samples and necessary buffers are placed into the instrument. A USB stick containing the information can be used to run a paired array and reagent plate placed into the instrument, giving the software the recipe for the assay and the specific reagent that can be spotted on each cluster. The MAVERICK can be contain a main controller board that runs the processes, a tunable light source or laser such as a continuously variable laser centered around 1550 nm, a beam splitter so that light can go into each of the 136 waveguides, an etalon that can be used as a reference, multiple photo detectors to capture ring and etalon signals, pumps to move the fluid from the reagent plate over the chip, and motors to move the reagent plate so the appropriate buffer can be available to the aspiration tubes.

### Determining Bioavailability of a Biologic

Some embodiments of the methods, systems and compositions provided herein include determining the bioavailability of a biologic in a subject. Advantageously, embodiments provided herein include the use of optical sensors which can provide a rapid and reliable test for the presence or absence of a biologic in a sample from a subject. Some embodiments can include providing a sample obtained from a subject. In some embodiments, the subject is mammalian, such as human. In some embodiments, the subject has been treated with the biologic. For example, the provided sample can be obtained from a subject that was previously administered the biologic more than 1 day, 3 days, 5 days, 7 days, 10 days, 12 days, 14 days, and 20 days, or any period of time in a range between any two of the foregoing periods, before the sample was obtained from the subject. In some embodiments, the subject has been previously administered the biologic more than 1 week, 2 weeks, 3 weeks, 4 weeks, 5 weeks, 7 weeks, 8 weeks, 9 weeks, 10 weeks, or any period of time in a range between any two of the foregoing periods, before the sample was obtained from the subject. In some embodiments, the sample comprises a serum sample from the subject.

The biologic can be a therapeutic composition. In some embodiments, a biologic can include an antigen-binding protein, such as an antibody, such as a monoclonal antibody, or antigen-binding fragment thereof. In some such embodiments, the biologic can include a human IgG polypeptide constant region such as a light chain, or a heavy chain, or fragment thereof. In some embodiments, the biologic can include a human IgG1 constant region, or fragment thereof. Examples of biologics useful with the embodiments provided herein include infliximab, abatacept, adalimumab, alefacept, etanercept, trastuzumab, ustekinumab, golimumab, and certolizumab pegol.

Some embodiments of determining the bioavailability of a biologic in a subject can include the use of optical sensors, such as ring resonators. In some embodiments, a flow cell comprises the optical sensor. In some embodiments, a chip comprises the optical sensor. In some embodiments, a first capture probe can be attached to an optical sensor. The first capture probe can be attached to the optical sensor at a certain concentration. In some embodiments, the first capture probe can be spotted on the optical sensor. In some embodiments, the amount of the first capture probe attached to the optical sensor can be about 1 μg, 5 μg, 10 μg, 20 μg, 50 μg, 100 μg, 200 μg, 300 μg, 500 μg, or more, or any amount in a range between any two of the foregoing amounts. The first capture probe can selectively bind to a biologic. In some such embodiments, a first capture probe can be the therapeutic target of the biologic. In some embodiments, the biologic is capable of binding the first capture probe in vivo. Examples of a first capture probe include a protein such as TNFα, CD80, CD86, CD2, HER2, IL-12, IL-23, and a fragment of any one of the foregoing polypeptides which selectively binds to the biologic.

In some embodiments, a sample is contacted with the first capture probe, in which the first capture probe is attached to the optical sensor. The sample can contact the first capture probe for a period of time such as at least about 10 seconds, 20 seconds, 30 seconds, 40 seconds, 50 seconds, 60 seconds, 1 minute, 1.5 minutes, 2 minutes, 2.5 minutes, 3 minutes, 3.5 minutes, 4 minutes, 4.5 minutes, 5 minutes, or more, or a period in a range between any two of the foregoing periods. In some embodiments, the sample flows over the first capture probe. The flow can be continuous, or can be halted and restarted. In some embodiments, the rate of flow of the sample over the first capture probe can be about 1 μl/min, 5 μl/min, 10 μl/min, 15 μl/min, 20 μl/min, 25 μl/min, 30 μl/min, 35 μl/min, 40 μl/min, 45, 50 μl/min, 55 μl/min, 60 μl/min, 70 μl/min, 80 μl/min, 90 μl/min, 100 μl/min, or more, or a rate in a range between any two of the foregoing rates. In some such embodiments, a biologic can bind to the first capture probe.

In some embodiments, a signal from a biologic bound to the first capture probe can be amplified. In some such embodiments, a second capture probe is contacted with the biologic bound to the first capture probe. The second capture probe can selectively bind to the biologic. In some embodiments, the second capture probe selectively binds to a human antibody, a humanized antibody, or a fragment thereof. In some such embodiments, the second capture probe can include an anti-human IgG antibody or antigen-binding fragment thereof.

The second capture can contact a biologic bound to the first capture probe for a period of time such as at least about 10 seconds, 20 seconds, 30 seconds, 40 seconds, 50 seconds, 60 seconds, 1 minute, 1.5 minutes, 2 minutes, 2.5 minutes, 3 minutes, 3.5 minutes, 4 minutes, 4.5 minutes, 5 minutes, or more, or a period in a range between any two of the foregoing periods. In some embodiments, the second capture probe flows over the biologic bound to the first capture probe. The flow can be continuous, or can be halted and restarted. In some embodiments, the rate of flow of the sample over the first capture probe can be about 1 μl/min, 5 μl/min, 10 μl/min, 15 μl/min, 20 μl/min, 25 μl/min, 30 μl/min, 35 μl/min, 40 μl/min, 45, 50 μl/min, 55 μl/min, 60 μl/min, 70 μl/min, 80 μl/min, 90 μl/min, 100 μl/min, or more, or a rate in a range between any two of the foregoing rates.

In some embodiments, after contacting the first capture probe with the sample, the first capture probe and the optical sensor can be washed by flowing buffer over the first capture probe and the optical sensor. In some embodiments, after contacting the biologic bound to the first capture probe with the second capture probe, the biologic bound to the first capture probe and the optical sensor can be washed by flowing buffer over to the first capture probe and the optical sensor and the optical sensor. For example, a wash step can include flowing buffer over at least the optical sensor for a period of time such as at least about 10 seconds, 20 seconds, 30 seconds, 40 seconds, 50 seconds, 60 seconds, 1 minute, 1.5 minutes, 2 minutes, 2.5 minutes, 3 minutes, 3.5 minutes, 4 minutes, 4.5 minutes, 5 minutes, or more, or a period in a range between any two of the foregoing periods. The flow can be continuous, or can be halted and restarted. In some embodiments, the rate of flow of the buffer over at least the optical sensor can be about 1 μl/min, 5 μl/min, 10 μl/min, 15 μl/min, 20 μl/min, 25 μl/min, 30 μl/min, 35 μl/min, 40 μl/min, 45, 50 μl/min, 55 μl/min, 60 μl/min, 70 μl/min, 80 μl/min, 90 μl/min, 100 μl/min, or more, or a rate in a range between any two of the foregoing rates.

In some embodiments, resonance wavelengths at the optical sensor can be measured and a change, no substantial change, or no change in the resonance wavelengths at the optical sensor can be indicative of the presence or absence of the biologic in the sample. In some embodiments, no substantial change in the resonance wavelengths at the optical sensor can be a change less than 20%, 15%, 10%, 5%, 4%, 3%, 2%, 1%, 0.5% 0.1% or any percentage change between any two of the foregoing percentages. In some embodiments, no substantial change in the resonance wavelengths at the optical sensor can be a change within a standard error for measuring a signal in the resonance wavelengths at the optical sensor. In some embodiments, no substantial change in the resonance wavelengths at the optical sensor can be a change less than a change within a range of the level of background noise above a signal and the level of the signal for the measurement of a resonance wavelength at the optical sensor. In some embodiments, no substantial change in the resonance wavelengths at the optical sensor can be less than any of 20%, 15%, 10%, 5%, 4%, 3%, 2%, 1%, 0.5%, or 0.1% and greater than 0% such that no substantial change in the resonance wavelengths at the optical sensor can correspond to any range between any of these values, 20%, 15%, 10%, 5%, 4%, 3%, 2%, 1%, 0.5%, and 0%. In some such embodiments, the biologic can have bioavailability for the subject. In some embodiments, no substantial change, or no change, in resonance wavelengths at the optical sensor can be indicative of the absence of the biologic in the sample. In some such embodiments, the biologic can have no substantial or no bioavailability for the subject. Without wishing to be bound by one theory, in some such embodiments, the subject may have antibodies against the biologic which prevent the biologic from binding to the first capture probe, and/or compete with the first capture probe for binding to the biologic. In some embodiments, the biologic can be cleared or degraded by the subject such that no substantial change in or no change, in resonance wavelengths at the optical sensor is measured.

Some embodiments can also include other assays to determine the presence or absence of an antibody against the biologic in a sample from a subject. Some such assays can include a third capture probe which selectively binds to a biologic or to the second capture probe, and has a first binding partner. A sample from the subject can be contacted with the third capture probe, and any complexes comprising the biologic bound to the third capture probe can be removed from the sample using a fourth capture probe with a second binding partner that binds to the first binding partner. Examples of binding partners include biotin and streptavidin. The removed complex can then be characterized for the presence or absence of an antibody against the biologic.

### Systems for Detecting a Biologic

Some embodiments of the methods, systems and compositions provided herein include systems for detecting the presence or absence of a biologic in a sample. Some such embodiments can include an optical sensor comprising: a ring resonator, a first capture probe attached to the ring resonator, wherein the first capture probe selectively binds to a therapeutic biologic. Some embodiments can also include a second capture probe which selectively binds to the biologic, and a detector adapted to measure a change, no substantial change, or no change in one or more resonance wavelengths at the optical sensor. In some embodiments, no substantial change in the resonance wavelengths at the optical sensor can be a change less than 20%, 15%, 10%, 5%, 4%, 3%, 2%, 1%, or any percentage change between any two of the foregoing percentages. In some embodiments, no substantial change in the resonance wavelengths at the optical sensor can be a change within a standard error for measuring a signal in the resonance wavelengths at the optical sensor. In some embodiments, no substantial change in the resonance wavelengths at the optical sensor can be a change less than a change within a range of the level of background noise above a signal and the level of the signal for the measurement of a resonance wavelength at the optical sensor. In some embodiments, no substantial change in the resonance wavelengths at the optical sensor can be less than any of 20%, 15%, 10%, 5%, 4%, 3%, 2%, 1%, 0.5%, or 0.1% and greater than 0% such that no substantial change in the resonance wavelengths at the optical sensor can correspond to any range between any of these values, 20%, 15%, 10%, 5%, 4%, 3%, 2%, 1%, 0.5%, and 0%. Additional information about ring resonator optical sensors and associated detection methods can be found in the following references: U.S. Pat. Nos. 9,846,126, 9,921,165, 9,983,206, U.S. Pat Pub. No. 2011/0045472, and U.S. Pat Pub. No. 2013/0261010, each of which is hereby incorporated by reference herein in its entirety.

Some embodiments also include an assay for determining the presence of an antibody against the therapeutic biologic in a sample, the assay can include a third capture probe which selectively binds to the therapeutic biologic or to the second capture probe, wherein the third capture probe comprises a first binding partner; and a fourth capture probe which selectively binds to the third capture probe, wherein the fourth capture robe comprises a second binding partner. In some embodiments, the first or second binding partner is selected from biotin and streptavidin. Example assays for detecting autoantibodies against a therapeutic biologic are disclosed in U.S. Pat Pub. No. 20170176433 which is incorporated by reference in its entirety.

### Therapeutic Embodiments

Some embodiments of the methods and compositions provided herein relate to a method for monitoring and/or optimizing therapy to a biologic in a subject receiving a course of therapy with the biologic. In some embodiments, the method can include (a) detecting or measuring the presence, level, or percent of the biologic at a plurality of time points over the course of therapy; (b) detecting a change in the presence, level, or percent of the biologic over time; and (c) determining a subsequent dose of the course of therapy for the subject or whether a different course of therapy should be administered to the subject based upon the change in the presence, level, or percent of the biologic over time. In some embodiments, the plurality of time points comprises at least 2, 3, 4, 5, 6, 7, 8, 9, 10, 11, 12, 13, 14, 15, 16, 17, 18, 19, 20, 25, 30, 35, 40, 45, 50, or more time points.

In some embodiments, a method for monitoring and/or optimizing therapy to a biologic in a subject receiving a course of therapy with the biologic can include: (a) measuring the level or percent of the biologic in a first sample from the subject at time point t₀; (b) measuring the level or percent of the biologic in a second sample from the subject at time point t₁; (c) optionally repeating step (b) with n additional samples from the subject at time points tₙ₊₁, wherein n is an integer from 1 to about 25 (e.g., n is 1, 2, 3, 4, 5, 6, 7, 8, 9, 10, 11, 12, 13, 14, 15, 16, 17, 18, 19, 20, 21, 22, 23, 24, or 25, or any range therein); (d) detecting a change in the level or percent of the biologic from time points t₀ to t₁ or from time points t₀ to tₙ₊₁; and (e) determining a subsequent dose of the course of therapy for the subject or whether a different course of therapy should be administered to the subject based upon the change in the level or percent of the biologic over time.

In certain other embodiments, the level or percent of the biologic is measured during the course of therapy at one or more (e.g., a plurality) of the following weeks: 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 52, 54, 56, 58, 60, 62, 64, 66, 68, 70, 80, 90, 100, etc.

In some embodiments, determining a subsequent dose of the course of therapy for the subject comprises maintaining, increasing, or decreasing a subsequent dose of the course of therapy for the subject. In other embodiments, determining a different course of therapy for the subject comprises treatment with a different biologic drug. In other embodiments, determining a different course of therapy for the subject comprises treatment with the current course of therapy along with another therapeutic agent. In further embodiments, determining a different course of therapy for the subject comprises changing the current course of therapy (e.g., switching to a different biologic or to a drug that targets a different mechanism).

In some embodiments, an increase in the level or percent of the biologic over time is an indication that treatment adjustment should be recommended for the subject. In some embodiments, a change from an absence of the biologic to the presence thereof over time is an indication that treatment adjustment should be recommended for the subject. In such embodiments, the subject can be treated with the current course of therapy (e.g., taking the existing biologic) along with one or more other therapeutic agents. In some embodiments embodiments, the subject can be switched to a different biologic. In some embodiments, the subject can be switched to a drug, such as biologic and/or non-biologic, that targets a different mechanism.

### EXAMPLES

### Example 1—Preparation of TNFα-Spotted Chips

Waveguides on silicon chips were functionalized with human TNFα (TNA-H4211, Acro Biosystems). TNFα was spotted on to waveguides of chips at various concentrations by a method substantially similar to the following method.

Prior to spotting, the chips were placed into an anodized chip rack for processing. The chips went through a series of washes for 2 minutes each: acetone to remove the photoresist that protects the chip surface, amino silane in acetone,(3-aminopropyltriethoxysilane, Thermo Scientific, Waltham, Mass.) to cover the chip surface with a uniform layer of functional amino groups, and isopropanol followed by water to wash off excess reagents prior to spotting. The chips were then dried using nitrogen gas and transferred onto the spotting deck. Three drops of about 300 μl of bis-sulfosuccinimidyl suberate (BS3) (Thermo Scientific, Waltham, Mass.), an amino-amino homobifunctional cross linker was placed on the desired rings and allowed to completely dry prior to continuing. A further 3 drops of about 300 μl of TNFα at 200 μg/ml in Drycoat plus 1% sucrose was then spotted onto the clusters that were activated with BS3. This was <0.2 ng/spot of antigen. After all analytes were spotted, the chips were allowed to dry for 1 hour in the humidity chamber. The chips were stored in a sealed, desiccated, nitrogen filled foil bag. Chips were stored at 2-8° C. before and after assembly into a Maverick consumable which includes a gasket and aspiration tubes.

### Example 2—Detection of Anti-TNFα Antibodies

Human serum samples were tested for the presence of therapeutic anti-TNFα antibody in patients treated with Infliximab (REMICADE) or Adalimumab (HUMIRA). Infliximab and Adalimumab are each a therapeutic anti-TNFα antibody. The assay was substantially similar to the following method.

For the anti-TNFα antibody assay, the serum sample was diluted 1:50 in running buffer. The chip was equilibrated by flowing running buffer over the chip for 1.5 min at 40 μl/min in order to get a base resonance frequency for each ring, followed by diluted sample for 3 minutes at 40 μl/min, then a wash step with running buffer for 2 minutes at 40 μl/min, followed by the amplification step for 3 minutes at 30 μl/min. The 2 baselines were determined by averaging the wavelength of ring resonance during the last 30 seconds of the wash and the last 30 seconds of the amplification step. Thus, the entire assay took less than 10 minutes per chip. Since the chip was in the reading head of the instrument for the entire assay, an array of 12 chips took about 2 hours to run. The signal was the shift from the first baseline to the second baseline. Amplification with anti-IgG Amplification with anti-IgG was done for two reasons. In complex matrices such as serum and whole blood, molecules other than IgG may bind to the antigen, so the initial shift was not specific. Besides increasing specificity, the anti-IgG amplified the initial signal from specific IgG binding to the antigen.

Using methods substantially similar to the foregoing methods, human serum samples from patients that had not been treated with an anti-TNFα medication were tested with waveguides that had been functionalized with various concentrations of TNFα. No signals were observed from the waveguides contacted with serum samples from patients that had not been treated with an anti-TNFα medication, such as Infliximab or Adalimumab. The results are summarized in TABLE 1.

TABLE 1

| TNFα concen- | Signal (relative units) | |
|---|---|---|
| tration (μg/ml) | Sample A (#453) | Sample B (#454) |
| 10 | −25 | −21 |
| 25 | −22 | −21 |
| 50 | −22 | −22 |
| 75 | −20 | −21 |
| 100 | −19 | −19 |
| 150 | −18 | −19 |
| 200 | −18 | −17 |
| 250 | −16 | −17 |
| 300 | −16 | −15 |

Human serum samples from patients that had been treated with Infliximab (Samples C and D) or Adalimumab (Samples E and F) were tested with waveguides that had been functionalized with various concentrations of TNFα. The results are summarized in TABLE 2. Signals were observed for serum samples from Samples C, D and F; however, a substantially reduced signal was observed from Sample E.

TABLE 2

| TNFα concen- | Signal (relative units) | | | |
|---|---|---|---|---|
| tration | Sample C | Sample D | Sample E | Sample F |
| (μg/ml) | (#451) | (#455) | (#456) | (#457) |
| 10 | −6 | −9 | −21 | −18 |
| 25 | 26 | 10 | −21 | −12 |
| 50 | 145 | 87 | −20 | 15 |
| 75 | 298 | 220 | −12 | 84 |
| 100 | 403 | 317 | −8 | 149 |
| 150 | 486 | 395 | −3 | 207 |
| 200 | 520 | 433 | 4 | 254 |
| 250 | 518 | 433 | 4 | 258 |
| 300 | 514 | 426 | 6 | 273 |

Serum samples were tested for the presence of anti-drug antibodies (anti-Adalimumab antibodies). Briefly, serum samples were contacted with biotinylated Adalimumab, complexes that included the biotinylated Adalimumab were captured with streptavidin, and any antibodies associated with the biotinylated Adalimumab were characterized. Serum sample E contained anti-drug antibodies (anti-Adalimumab antibodies) which produced the substantially reduced signal.

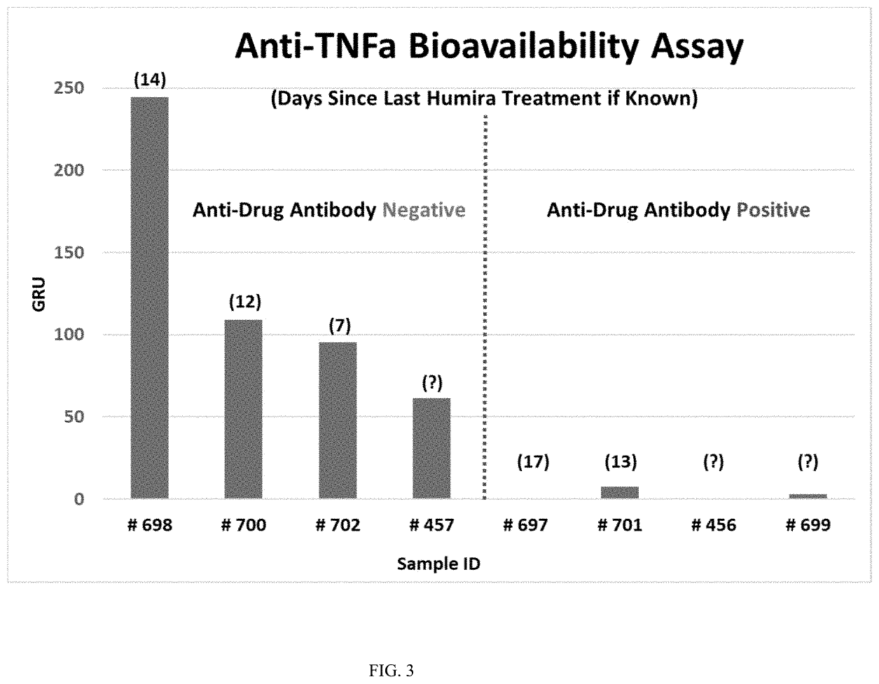

Various serum samples were tested for binding to waveguides functionalized with TNFα, and for the presence of anti-drug antibodies (anti-Adalimumab antibodies). The results are shown in FIG. 3. Signals from the waveguides were observed for serum samples from patients treated with an anti-TNFα therapy and that tested negative for anti-drug antibodies. In contrast, serum samples from patient treated with an anti-TNFα therapy and that tested positive for anti-drug antibodies had a substantially reduced signal from the waveguides functionalized with TNFα.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of determining the bioavailability of a biologic in a subject comprising:
(a) providing a sample obtained from a subject treated with a biologic;
(b) contacting a first capture probe attached to an optical sensor with the sample, wherein the biologic selectively binds to the first capture probe;
(c) contacting the biologic bound to the first capture probe with a second capture probe, wherein the second capture probe selectively binds to the biologic; and
(d) measuring a change, no substantial change, or no change in one or more resonance wavelengths at the optical sensor, thereby detecting the presence or absence of the biologic in the sample.

2.-37. (canceled)
